(19) United States
(12) Patent Application Publication
(10) Pub. No.: US 2010/0222203 A1
(43) Pub. Date: Sep. 2, 2010

(54) PRODUCTION METHOD FOR PROPYLENE, RESTORATION METHOD FOR CATALYST, AND SOLID ACID CATALYST

(75) Inventors: Toshihide Baba, Kanagawa-ken (JP); Reiko Ono, Aichi-ken (JP); Nobutaka Homma, Shizuoka-ken (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: TOYOTA JIDOSHA KABUSHIKI KAISHA, Toyota-shi, Aichi-ken (JP); TOYOTA TSUSHO CORPORATION, Nagoya (JP)

(21) Appl. No.: 12/161,218

(22) PCT Filed: Jan. 18, 2007
(86) PCT No.: PCT/IB2007/000191

§ 371 Date: Jul. 17, 2008

(30) Foreign Application Priority Data
Jan. 20, 2006 (JP) 2006-012634

(51) Int. Cl. B01J 38/02; C07C 1/20; B01J 29/06

(52) U.S. Cl. 502/56; 585/639; 585/324; 502/60

(57) ABSTRACT

A production method for propylene is characterized in that ethanol is converted into propylene by continuously reacting ethanol on a catalyst. A solid acid catalyst is characterized in that the kinetic constant k of the butane cracking reaction on the catalyst at 500° C. is 0.1 to 30 (cm³/min·g), and the solid acid catalyst is used in the production method for propylene. A solid acid catalyst is characterized in that the aperture diameter of pores formed in surfaces of the catalyst is 0.3 to 1.0 nm, and the solid acid catalyst is used in the production method for propylene. Furthermore, a regeneration method for a catalyst is characterized in that a heating treatment in an oxygen atmosphere is perform on a catalyst that has been used to produce propylene in the production method for propylene of the invention.

### BACKGROUND OF THE INVENTION

1. Field of the Invention

The invention relates to a method for producing propylene, and a solid acid catalyst that is used in the production method as well as a method for regenerating the catalyst used in the production method.

2. Description of the Related Art

The demand for propylene, a principal raw material in petrochemistry, is expected to rapidly grow in the future. In order to increase the production of propylene, various production methods have been described, for example, in Japanese Patent Application Publication No. 2003-326169 (hereinafter “JP-A-2003-326169”), and PETROTEC Vol. 27, No. 8, p 628-632 (2004) (hereinafter “PETROTEC”), etc.

JP-A-2003-326169 describes a catalyst in which a metal, such as nickel or the like, is supported on a regular mesoporous body having a pore size from 2 to 10 nm. Then, in a gas phase, propylene or the like can be selectively produced from ethylene. PETROTEC describes an example in which a solid acid catalyst, supported with nickel, is used to form propylene from ethylene. In particular, the example described in PETROTEC uses the solid acid catalyst H-ZSM-5 (proton exchange type ZSM-5).

However, a generally-used reaction mechanism based on the metathesis reaction between ethylene and 2-butene has a problem of low propylene selectivity. Furthermore, because the starting raw material is a gas (ethylene), the storage, handling, and transportation thereof is troublesome.

### SUMMARY OF THE INVENTION

The invention provides a method for producing propylene that is high in the propylene selectivity, and allows the use of a material that is easier to handle, and is capable of producing propylene at high selectivity. The invention also provides a solid acid catalyst that is suitable for the propylene production method. Furthermore, the invention provides a method for regenerating the catalyst that efficiently regenerates the catalyst used in the production method.

In the method for producing propylene according to the invention, ethanol is converted into propylene by continuously reacting ethanol on a catalyst.

The aforementioned “continuously reacting” refers to a first-stage conversion of ethanol to propylene, or a conversion from ethylene, which is a substance formed by an intramolecular dehydration reaction of ethanol, into propylene. These reaction mechanism of the conversions are differs from the mechanism of forming propylene through a carbene intermediate in a conventional metathesis reaction.

In the production method, the ethanol may be converted into the propylene by: forming ethylene through a dehydration reaction between the catalyst and the ethanol; forming a butyl cation through a dimerization reaction between the ethylene formed and an ethyl cation obtained from the catalyst; forming a butene from the butyl cation; forming a carbocation through a trimerization reaction between the butene and the ethyl cation; and forming propylene through a cleavage reaction of the carbocation.

The catalyst used in the method for producing propylene may be a solid acid catalyst. In the production method, a fixed-bed flow type reaction system is used, and the reaction progresses via carbocation reaction intermediates. Therefore, a solid acid catalyst with protons present on the catalyst surface is suitable in the method.

A kinetic constant of the butane cracking reaction on the solid acid catalyst may be 0.1 to 30 (cm³/min·g). With the activity coefficient defined within the aforementioned range, excessive oligomer reactions are unlikely to occur, and the propylene selectivity is improved.

In particular, the solid acid catalyst may be a zeolite. Herein, the “zeolite” is a hydrous alumino-based silicate having molecular-level pores, and may be represented by the general formula: M_{2/n}O.Al₂O₃.xSiO₂.yH₂O. There are various zeolites in IUPAC classification indicating crystalline structures, and mineral names in accordance with different compositions. The type of zeolite used in the invention is not limited to any particular type, i.e., any zeolite may be used. For example, A-type zeolite, L-type zeolite, X-type zeolite, Y-type zeolite, β-type zeolite, ferrierite-type zeolite, mordenite-type zeolite, FMI-type zeolite (ZSM-5 zeolite), phosphate-based zeolite, etc. may be used. In addition, the solid acid catalyst of the invention may be used not only in the form of one species of catalyst, but also in combination of a plurality of species of catalysts. The solid acid catalyst may be in the form of powder or granules, and may also be a catalyst obtained by supporting a solid acid catalyst on a support (porous substance) surface.

Taking the rate of conversion to propylene into consideration, the solid acid catalyst may be a phosphate-based zeolite. Herein, the phosphate-based zeolite has a structure in which some of the Si atoms of SiO₄ tetrahedrons of zeolite are substituted by trivalent Al (aluminum) atoms and pentavalent P (phosphorus) atoms, and furthermore, some of the P or Al atoms are substituted by metal cations of different valances in order to give solid acidity.

Concrete examples thereof include silicon aluminophosphates (SiAlPO₄-n) with some of the P atoms substituted by Si; metal aluminophosphates (MeAlPO₄-n) with Al atoms substituted by metal cations of different valences; and intermediate compositions (MeAlPSiO₄-n) between these aluminophosphates (“n” in the parentheses is a number indicating a crystal structure). More concretely, SiAlPO-5, SiAlPO-11, SiAlPO-18, SiAlPO-34, CoAlPO-5, CoAlPO-34, CoAlPO-34, BeAlPO-5, BeAlPO-34, BeAlPSO, MnAlPO-34, MnAlPO-36, etc. may be used.

If a phosphate-based zeolite is used as the solid acid catalyst, the reaction temperature may be 300 to 500° C. A reaction temperature of 300 to 500° C. will make it possible to maintain a high rate of selection to propylene while maintaining a fast velocity of conversion to ethylene. The reaction temperature may be 350 to 470° C. Furthermore, the reaction temperature may be 400 to 450° C.

As for the solid acid catalyst, at least one species of metal ion selected from the group consisting of alkali metals, alkaline-earth metals, Group 5 metals, Group 8 metals, Group 10 metals, Group 11 metals and Group 12 metals may be supported (doped) therein. Using these metal ions, the amount of protons in the catalyst can be adjusted to perform reaction control. Consequently, the propylene selectivity may be improved. If such a catalyst with metal ions introduced is used, the reaction temperature may be 300 to 500° C.

Alternatively, ions of calcium, lead and zinc may also be used as the metal ion. In this case, the reaction temperature may be 300 to 500° C.

The aperture diameter of the pores formed in the solid acid catalyst may range from 0.3 to 1.0 nm. A narrower range of the aperture diameter of the pores that is acceptable is 0.3 to 0.8 nm. The range of the aperture diameter may also be as narrow as 0.3 to 0.5 nm. If the aperture diameter of the catalyst is approximately equal to the occupation diameter of propylene (about 0.4 nm), propylene, produced by the reaction, is selectively released from the interiors of pores of the catalyst, so that further reaction of propylene is inhibited. As a result, the propylene selectivity is improved. Incidentally, the aperture diameter of the pores may expand and shrink in accordance with the molecular vibrations based on the temperature during the reaction, and is not necessarily equal to the occupation diameter of propylene.

Any one of the following four methods may be employed as the method of producing propylene according to the invention. In the first production method, propylene is continuously produced by supplying ethanol to a reaction bed charged with at least one species of the solid acid catalyst (see FIG. 2A). In the second production method, the use of a catalyst vessel, constructed of at least two beds that are a reaction bed (dehydration catalyst bed) charged with a dehydration catalyst, to be described later, and a reaction bed (solid acid catalyst bed) charged with at least one species of the solid acid catalyst, propylene is continuously produced by supplying ethanol from the dehydration catalyst bed (see FIG. 2B). In the third production method, a series arrangement of a catalyst vessel having a reaction bed (dehydration catalyst bed) charged with a dehydration catalyst, to described later, and a catalyst vessel having a reaction bed (solid acid catalyst bed) charged with at least one species of the solid acid catalyst, propylene is continuously produced by causing ethanol to flow from the dehydration catalyst bed (see FIG. 2C). According to a fourth production method, a moisture removal bed (hereinafter “dehydration catalyst bed”), charged with a dehydration catalyst (to be described later), is provided downstream of the reaction bed and upstream of the reaction bed (hereinafter “solid acid catalyst bed”), charged with at least one species of the solid acid catalyst, propylene is continuously produced by supplying ethanol from the dehydration catalyst bed (see FIG. 2D).

The dehydration catalyst refers to a catalyst that forms ethylene from ethanol through an intramolecular dehydration reaction. That is, the solid acid catalyst of the invention may be a dehydration catalyst. In addition, a catalyst generally for use for producing ethylene from ethanol, such as active alumina or the like, may also be used. Furthermore, a combination of the solid acid catalyst of the invention and a general dehydration catalyst may also be used. Furthermore, the dehydration catalyst may be in the form of a powder or a grain, and may also be a catalyst obtained by supporting a dehydration catalyst on a support (porous substance) surface. As for the configuration of the dehydration catalyst bed used in the second to fourth production methods, a dehydration catalyst charged in a vessel is provided, and an inlet for supplying ethanol into the vessel and an outlet for discharging the reaction products (ethylene or the like) from the dehydration catalyst are provided. The dehydration catalyst bed may be either of a fixed bed type, in which the dehydration catalyst in the vessel remains fixed at the time of supply of raw materials, or of a fluid bed type in which the dehydration catalyst moves due to the flow of raw materials when the raw materials are supplied.

As for the configuration of the solid acid catalyst bed used in the first to fourth production methods, a solid acid catalyst charged in a vessel is provided, and an inlet for supplying the reaction products (ethylene and the like) from the dehydration catalyst into the vessel, and an outlet for discharging the reaction products (propylene and the like) from the solid acid catalyst are provided. The solid acid catalyst bed may be either of a fixed bed type in which the solid acid catalyst in the vessel remains fixed at the time of supply of raw materials or of a fluid bed type in which the solid acid catalyst moves due to flowage of raw materials when the raw materials are supplied. In addition, the “inlet . . . is provided” or the “outlet . . . is provided” mentioned above in connection with the configuration of the dehydration catalyst bed and the configuration of the solid acid catalyst bed indicate the existence of a region that separates a pre-reaction process and a post-reaction process of each bed from each other. Referring to FIG. 2B, in the configuration of the dehydration catalyst bed used in the second production method, a region where ethanol enters the dehydration catalyst bed in the catalyst vessel serves as the “inlet for supplying ethanol”, and a region where ethylene and the like formed by the dehydration catalyst exits the dehydration catalyst bed serves as the “outlet for discharging the products resulting from reactions on the dehydration catalyst”.

In the second to fourth production methods, the reaction operations may be performed with different energies in the dehydration catalyst bed and the solid acid catalyst bed. In particular, a method in which a temperature gradient is provided in the catalyst; a method that uses irradiation with electron beams of different energies; etc. may be employed. In the construction used for the fourth production method, water generated through the dehydration reaction of ethanol may be removed, so that the propylene selectivity is improved. Thus, although a plurality of reaction beds is included, all the processes in producing propylene from ethanol may be continuously performed.

In the method for regenerating the catalyst according to the invention, the catalyst that in the above-described method for producing propylene is heated in an oxygen atmosphere. Generally, during the propylene formation reaction, coke or a coke precursor is deposited on the catalyst, which reduces the catalyst activity. By heating the catalyst in an oxygen atmosphere, the coke or the coke precursor on the catalyst is removed by burning, and catalytic activity is restored.

Furthermore, as for the solid acid catalyst of the invention, a kinetic constant k of a butane cracking reaction on the solid acid catalyst at 500° C. may be 0.1 to 30 (cm³/min·g), and the solid acid catalyst may be used in the above-described production method for propylene. If the kinetic constant k is 0.1 cm³/min·g or greater, sufficient reaction velocity for the conversion to propylene can be obtained. If the kinetic constant k is 30 cm³/min·g or less, occurrence of excessive oligomer reactions becomes unlikely, and the propylene selectivity can be improved.

With respect to the aperture diameter of pores formed in surfaces of the solid acid catalyst, appropriate aperture diameters may be 0.3 to 1.0 nm. The aperture diameter of pores may also be 0.3 to 0.8 nm, and furthermore, may be 0.3 to 0.5 nm. Then, the catalyst may be used in the foregoing production method for propylene. As the aperture diameter of the catalyst approaches the occupation diameter of propylene (about 0.4 nm), propylene is selectively released, after the reaction, from the interiors of pores of the catalyst, so that further change of propylene into other hydrocarbons is inhibited. As a result, the propylene selectivity can be improved. Incidentally, the aperture diameter of pores may expand and shrink in accordance with the molecular vibrations based on the temperature during the reaction, and is not necessarily equal to the occupation diameter of propylene.

The method for producing propylene according to the invention provides high propylene selectivity, and allows the use of raw materials with suitable handling characteristics, and is able to produce propylene at high selectivity. In addition, the solid acid catalyst of the invention is suitable for the propylene production method. Furthermore, the method for regenerating the catalyst efficiently regenerates the catalyst used in the production method.

### BRIEF DESCRIPTION OF THE DRAWINGS

The foregoing and/or further objects, features and advantages of the invention will become more apparent from the following description of preferred embodiment with reference to the accompanying drawings, in which like numerals are used to represent like elements and wherein:

FIG. 1 is a diagram showing the reaction scheme according to the invention;

FIGS. 2A to 2D are diagrams schematically showing reaction paths of the production methods for propylene according to the invention;

FIG. 3 is a diagram showing the relationship between the ethylene conversion rate and the propylene selectivity with regard to H-ZSM-5 catalyst with Pb ion introduced and H-ZSM-5 catalyst without Pb ion introduced;

FIG. 4 is a diagram showing the relationship between the ethanol and ethylene conversion rate and the propylene selectivity with regard to an SiAlPO-34 catalyst in the cases of varied contact times;

FIG. 5 is a diagram showing time-dependent changes in the ethylene conversion rate on the SiAlPO-34 catalyst at various temperatures;

FIG. 6 is a diagram showing time-dependent changes in the propylene selectivity on the SiAlPO-34 catalyst at various temperatures;

FIG. 7 is a diagram showing the relationship between the ethylene conversion rate and the product selectivity in the cases where the H-ZSM-5 catalyst is used and ethanol is a starting raw material (reaction temperature 320° C.);

FIG. 8 is a diagram showing the relationship between the ethylene conversion rate and the product selectivity in the cases where the H-ZSM-5 is used and a dehydration treatment is performed after the reaction of ethanol to ethylene (reaction temperature 320° C.); and

FIG. 9 is a diagram showing time-dependent changes in the ethylene conversion rate on a brand-new SiAlPO-34 catalyst and a post-regeneration treatment SiAlPO-34.

### DETAILED DESCRIPTION OF THE PREFERRED EMBODIMENTS

In the following description, the present invention will be described in more detail in terms of exemple embodiments.

A method of producing propylene will be described. The production method for propylene according to the invention converts ethanol into propylene by continuously reacting ethanol on a catalyst. By this method, propylene is efficiently produced from ethanol. In addition, because ethanol is a liquid at room temperature, it is easier to handle than ethylene, which is a gas at room temperature. Therefore, the cost of equipment may be reduced.

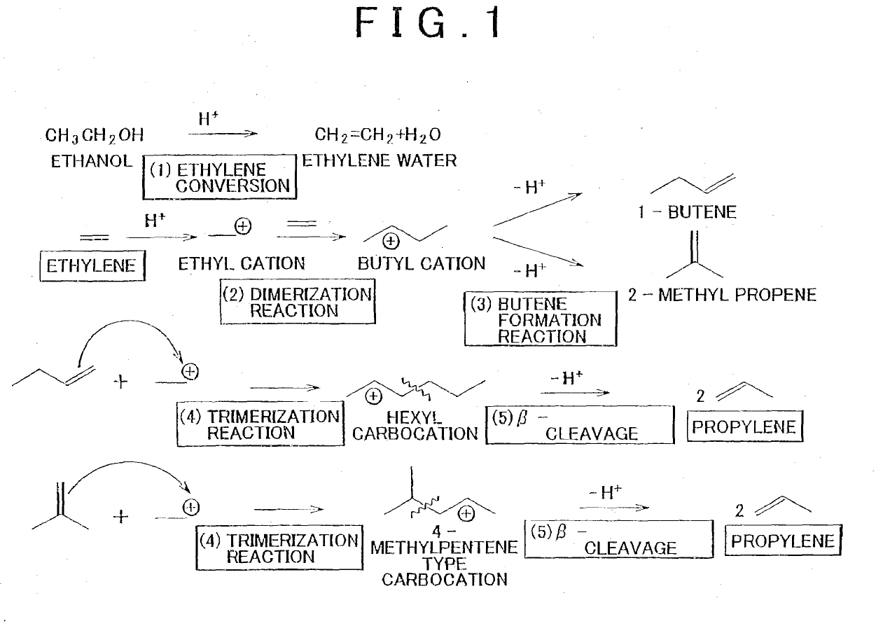

FIG. 1 shows a formation mechanism of propylene when a solid acid catalyst is used. In the formation mechanism, propylene is formed via a carbocation reaction intermediate. Specifically, as shown in FIG. 1, (1) first, ethylene is formed from ethanol through a dehydration reaction on an acid catalyst. (2) Next, the ethylene that is formed dimerizes with the ethyl cation provided by the acid catalyst (formation reaction of butyl cations). (3) Next, 1-butene or 2-methylpropene is formed from the butyl cations. (4) Next, the trimerization reaction due to the addition of ethyl cations to the two isomers of butene (formation reaction of (4-methylpentene type carbocations or hexyl carbocations) occurs. Finally, propylene is formed through a β-cleavage. As in the above, reactions continuously occur.

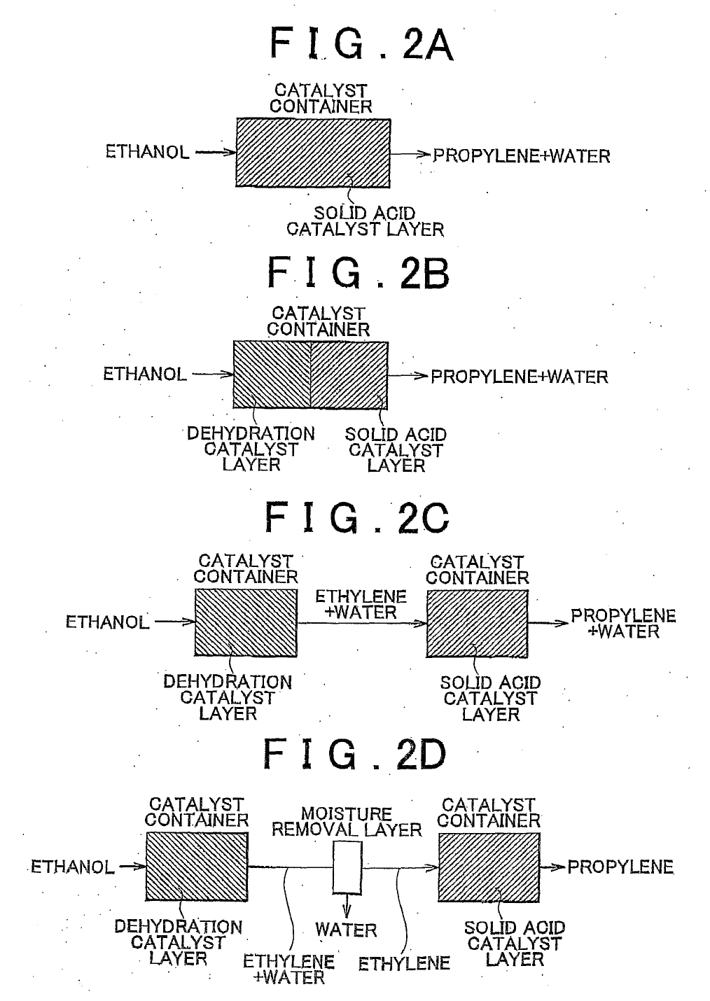

The method of producing propylene in the invention may be any one of the methods shown in FIGS. 2A-2D.

FIG. 2A shows a reaction path in which propylene is continuously produced by supplying ethanol to a reaction bed charged with at least one species of solid acid catalyst (hereinafter “solid acid catalyst bed”).

FIG. 2B shows a reaction path in which ethanol is continuously converted into propylene in a unitary catalyst vessel constructed of two beds. That is, a reaction bed charged with a dehydration catalyst (hereinafter “dehydration catalyst bed”) and a solid acid catalyst bed are both provided within the unitary vessel. Thus, in the method shown, propylene may be continuously produced by supplying ethanol to the dehydration catalyst bed.

FIG. 2C shows a reaction path in which ethanol is continuously converted into propylene in a construction made up of a serial arrangement of catalyst vessel having a dehydration catalyst bed and a solid acid catalyst bed. As in the method shown in FIG. 2B, propylene may be continuously produced by supplying ethanol to the dehydration catalyst bed.

FIG. 2D shows a reaction path in which ethanol is continuously converted into propylene in a construction in which a moisture removal bed is disposed between the dehydration catalyst bed and the solid acid catalyst bed. Propylene is produced as long as ethanol is supplied to the dehydration catalyst bed.

The aforementioned processes used in the production method for propylene according to the invention are obtained due to the discovery that the aforementioned solid acid catalyst accelerates the following three reactions, specifically, (1) the formation of propylene from ethanol, (2) the formation of ethylene from ethanol (dehydration reaction), and (3) the formation of propylene from ethylene.

The dehydration catalyst refers to a catalyst that forms ethylene from ethanol through an intramolecular dehydration reaction. That is, the solid acid catalyst of the invention may serve as a dehydration catalyst. In addition, a catalyst generally for use for producing ethylene from ethanol, such as active alumina or the like, may also be used. Furthermore, a combination of the solid acid catalyst of the invention and a general dehydration catalyst may also be used. The dehydration catalyst may be in the form of a powder or granules, and may also be a catalyst obtained by supporting a dehydration catalyst on support (porous substance) surfaces.

The moisture removal bed mentioned in the paragraph (D) removes water contained in the raw material and water formed on the dehydration catalyst. As the method of the paragraph (D), methods, such as liquefaction separation by cooling, separation by a water vapor-permeable membrane, etc., are may be used. In the method (B), (C) or (D), in the dehydration catalyst bed, which is upstream in a view from the ethanol supply side, ethanol is dehydrated to form ethylene, and the solid acid catalyst bed, which is downstream in the aforementioned view, propylene is formed from ethylene. While the reaction temperatures of the two catalyst beds may be equal or different, a constant pressure should be maintained throughout the processes so that the reactions from the stage of ethanol to formation of propylene proceed.

In each of the methods described above, the reaction temperature should be maintained at 300° C. to 500° C. when the solid acid catalyst, described later, is used. Maintaining the reaction temperature within the specified range will make it possible to inhibit polymerization reactions leading to compounds having larger molecular weights than propylene, and to curb excessive cracking reaction. Furthermore, in the methods (B), (C) and (D), the temperature of the dehydration catalyst bed should be 230° C. to 270° C. In the method (B), a temperature gradient may be established by setting the temperature of the ethanol supply side (corresponding to the dehydration catalyst bed) at 230° C. to 270° C., and setting the temperature of the reaction product outlet side (corresponding to the solid acid catalyst bed) at 300° C. to 500° C. Thus, even if a plurality of reaction beds are included as shown in the methods (B) to (D), the entire process of producing propylene from ethanol may be continuously performed.

Suitable solid acid catalysts include, for example, H-ZSM-5 (proton exchange type ZSM-5) or a phosphate-based zeolite (in particular, SiAlPO-34, SiAlPO-11 and SiAlPO-18 of the aforementioned zeolites). However, because of the resulting high propylene selectivity, and also considering easy regeneration and appropriate acid strength, phosphate-based zeolites are particularly suitable solid acid catalysts.

In addition, a phosphate-based zeolite may be used as a solid acid catalyst, and that the reaction temperature be 300° C. to 500° C. Adoption of such conditions will inhibit excessive polymerization reactions and will also inhibit excessive cracking reactions.

The solid acid catalyst will now be described. It is preferable that at least one species of metal ion selected from the group consisting of alkali metals, alkaline-earth metals, Group 5 metals, Group 8metals, Group 10 metals, Group 11 metals and Group 12 metals be doped into the solid acid catalyst that is represented by zeolites such as H-ZSM-5 and the like. Considering appropriate acid strength, it is preferable that at least one of calcium ion, lead ion and zinc ion be introduced therein. As a method of introduction, it is possible to apply a solid ion exchange process and the like.

As for the solid acid catalyst of the invention, the kinetic constant k of a butane cracking reaction on the solid acid catalyst at 500° C. is 0.1 to 30 (cm³/min·g). The constant k can be determined by a method described in “H. RASTELLI, Jr., B. M. LOK, J. A. DUISMAN, D. E. EARLS and J. T. MULLHAUPT, The Canadian Journal of Chemical Engineering, Vol. 60 (1982), p 44-49”.

Concretely, a helium gas containing 2% n-butane is passed to a catalyst (SiAlPO-34 or the like) that is kept at 500° C., and the amount of n-butane consumed in the gas released after the consumption by the butane cracking reaction is measured through gas chromatography. The obtained measurement value is substituted in the following expression to determine the constant k.

k=−(F/W)ln(1−c) (cm³/min·g)

In the expression, c is the molar fraction of the amount of n-butane consumed, and F is the flow rate of helium gas containing 2% n-butane (cm³/min), and W is the weight of the catalyst (g).

The constants k of H-ZSM-5, SiAlPO-34, and SiAlPO-11 are 65 to 70, 0.1 to 7.6, and 0.5 to 3.5, respectively.

In the solid acid catalyst of the invention, the aperture diameter of the pores is 0.3 to 1.0 nm. The molecular diameter (occupation diameter) of the butenes generated by the dimerization reaction shown in FIG. 1 is about 0.5 nm whereas the molecular diameter (occupation diameter) of propylene is about 0.4 nm, that is, the molecular diameter of propylene is smaller than that of the intermediate product. Therefore, by screening the products formed in catalyst pores by the size of the catalyst aperture diameter, the proportion of propylene released from the catalyst system (selectivity thereof) is improved. Incidentally, the aperture diameter of the pores expands and shrinks in accordance with the molecular vibrations based on the temperature during the reaction, and is not necessarily equal to the occupation diameter of propylene.

The pore diameter of ZSM-5 is about 0.6 nm, and the pore diameter of SiAlPO-34 is about 0.4 nm. Because the pore diameter of SiAlPO-34 is substantially equal to the molecule diameter of propylene, the “molecular sieve effect” of the catalyst aperture diameter increases the amount of propylene that is desorbed from interior of the catalyst pore.

The aperture diameter of pores may be measured by a known method. For example, the aperture diameter of pores may be determined by a pore measurement based on the adsorption process of a gas such as nitrogen gas, carbon dioxide, etc., or an electron microscopic measurement.

The method for regenerating the catalyst will now be described. In the regeneration method for the catalyst according to the invention the catalyst used to produce propylene by the above-described propylene production method is heated in an oxygen atmosphere. The catalytic activity is restored by burning and removing coke or coke precursors on the catalyst that are believed to be the cause of activity degradation.

A suitable heating temperature (firing temperature) for the heating treatment is 600 to 700° C. where the coke or the coke precursor thoroughly burns. The oxygen atmosphere may be established by introducing the atmospheric air into the catalyst vessel. Incidentally, this simple and easy regeneration method is particularly suitable for a catalyst be applied to an oxide catalyst according to the invention that has heat resistance. Such catalysts include, for example, SiAlPO-34, SiAlPO-11, SiAlPO-5 and SiAlPO-18.

Hereinafter, as examples of the invention, tests of production of propylene using of a metal-doped H-ZSM-5 catalyst will be described. A metal-doped H-ZSM-5 catalyst (amounts of each metal ion introduced: M/Al (molar ratio)=0.17 to 0.5, where M is the molar amount of the metal ion, and Al is the molar amount of aluminum ion) was prepared by doping an H-ZSM-5 catalyst (made by Tosoh Corporation) with SiO₂/Al₂O₃=23.8, Pb ion, Ca ion, Zn ion, Ag ion, Na ion, In ion, Ga ion and Ta ion, respectively, by a solid ion exchange process. As precursors of Pb ion, Ca ion, Zn ion, Ag ion, Na ion, In ion, Ga ion and Ta ion, nitrates thereof were used. As a starting raw material, ethylene was used. Incidentally, because ethanol is easily converted into substantially 100% ethylene (dehydration reaction by the solid acid catalyst) when supplied to the solid acid catalyst bed in a condition of 230° C. or higher, the use of ethylene a, a starting raw material still allows evaluation of the activity of the solid acid catalyst in the continuous production of propylene from ethanol. Therefore, the example corresponds to a reaction by the solid acid catalyst after the passage through the dehydration catalyst bed and the moisture removal bed shown in FIG. 2D.

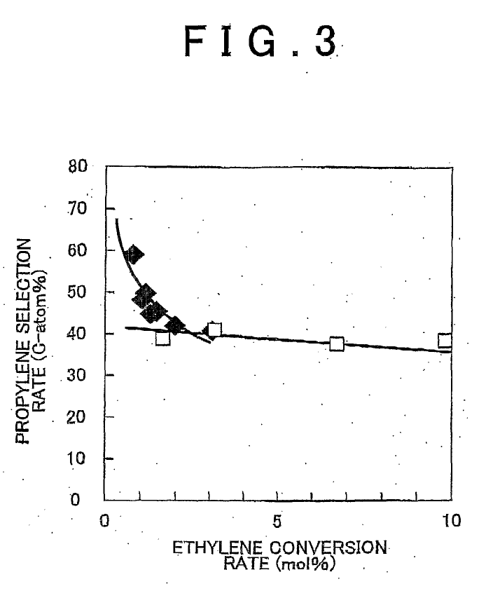

The reaction conditions in the production test are as follows. The substance amount ratio of the doped metal ion to Al in H-ZSM-5 (molar ratio: “molar amount of introduced metal ion”/“molar amount of aluminum ion”) is 0.5. The pressures of ethylene gas, a N₂ carrier gas, and a He carrier gas are each 33.8 kPa. The contact time is 0.45 to 1.8 (g-catalyst·h/mol). The reaction temperature is 325° C. FIG. 3 shows results of determination of the ethylene conversion rate and the propylene selectivity with contact times. The figure shows results from Pb/H-ZSM-5 having Pb ion as an introduced metal ion, and results from H-ZSM-5.

The introduction of a metal ion reduces the acid strength of the proton of the H-ZSM-5 catalyst. In addition, as shown in FIG. 3, extrapolation of the ethylene conversion rate to 0 mol % makes it possible to evaluate the influence of the metal ion on the propylene selectivity. Results (extrapolation values) obtained in the cases of various metal ions introduced are shown in Table 1.

TABLE 1

| Introduced | Propylene selectivity in |
|---|---|
| metal ion | extrapolation value (C-atom %) |
| None | 42 |
| Pb | 72 |
| Ca | 48 |
| Zn | 46 |
| Ag | 44 |
| Na | 42 |
| In | 41 |
| Ga | 40 |
| Ta | 37 |

With regard to the H-ZSM-5 catalyst, the propylene selectivity in the case of extrapolation of the ethylene conversion rate to 0 mol % was about 40 C-atom % (C-atom %: proportion of the number of carbon atoms changed into propylene to the total number of carbon atoms in the starting raw material). In contrast, the results for the H-ZSM-5 catalyst with various metal ions introduced show that the catalysts with Ca ion, Pb ion and Zn ion each exhibited a propylene selectivity as high as 48 to 72 C-atom % in the extrapolation of the ethylene conversion rate to 0 mol %, as shown in FIG. 3 and Table 1.

Hereinafter, the production test of propylene in which a phosphate-based catalyst was used will be described. This phosphate-based zeolite catalyst (SiAlPO-34) has a lower acid strength than the aforementioned H-ZSM-5 catalyst.

The SiAlPO-34 was prepared using the method described in “A. M. Prakash and S. Unnikrishnan, J. CHEM. SOC. FARADAY. TRANS., 1994, 90 (15), 2291-2296”.

Concretely, compounding was conducted so as to achieve the following proportion: Al₂O₃:P₂O₅:SiO₂:H₂O:morpholine=1:1:0.5:60:2. The compounding was conducted by adding distilled water to 85% orthophosphoric acid, and then adding alumina thereto, and stirring the mixture for 6 hours. Separately, fumed silica and morpholine, placed in distilled water, were obtained, and the aforementioned compound was dropped thereinto while the mixture was being strongly stirred. Then, the prepared gelatinous substance was aged for 24 hours at 27° C. After that, the substance was transferred into a heat-resistant container, and a hydrothermal synthesis reaction was conducted for 120 hours at 190° C. After being cooled, the substance was washed with water, the water decanted, and then dried to obtain an object substance (SiAlPO-34).

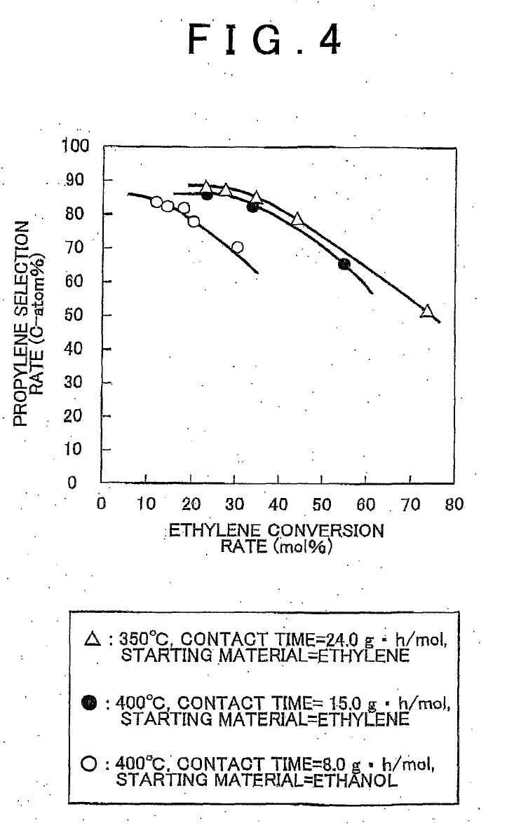

The reaction conditions in the production test are as follows. The following condition is the same in the cases where either ethanol or ethylene gas is used as a starting raw material. The pressures of ethanol or ethylene gas and a N₂ carrier gas and a He carrier gas were each set at 33 kPa, and the contact time was set at 3.6 to 24.0 (g-catalyst·h/mol), and the reaction temperature was set at 350 to 400° C. Test results are shown in FIG. 4. Incidentally, ethanol is easily converted into substantially 100% ethylene (dehydration reaction by the solid acid catalyst) when supplied to a solid acid catalyst bed having a temperature of 230° C. or higher. Therefore, the horizontal axis in FIG. 4 represents the ethylene conversion rate. In addition, in the example, the results from the test using ethylene as a starting raw material correspond to the reaction by the solid acid catalyst after the passage through the dehydration catalyst bed and the moisture removal bed shown in FIG. 2D.

As is apparent from FIG. 4, the propylene selectivity relative to the ethanol and ethylene conversion rate is markedly higher than in comparison with the H-ZSM-5 catalyst. Specifically, whereas the propylene selectivity of the H-ZSM-5 catalyst is 42 C-atom %, the propylene selectivity of the SiAlPO-34 catalyst is 83 to 87 C-atom %. A conceivable reason for this is that due to the lower acid strength of the SiAlPO-34 than that of the H-ZSM-5 catalyst, the formation of hydrocarbons with high carbon numbers is unlikely, and that the “molecular sieve effect” occurs. Incidentally, in the H-ZSM-5 catalyst in which a metal ion is introduced (Pb-introduced H-ZSM-5 catalyst or the like), an increase in the ethylene conversion rate sharply reduces propylene selectivity (see FIG. 3), whereas in the SiAlPO-34 catalyst, an increase in the ethylene conversion rate has a negligible effect on propylene selectivity

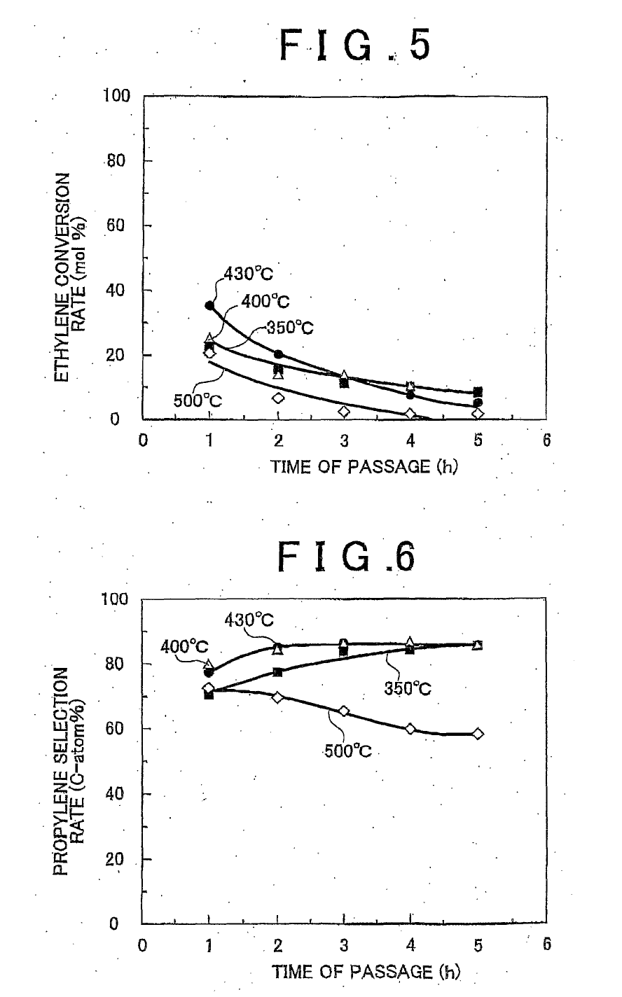

Hereinafter, the production test of propylene at various temperatures will be described. The production test of propylene was conducted by using SiAlPO-34 as a catalyst and changing the reaction temperature. Ethylene was used as a starting raw material. The reaction conditions of the production test are as follows. The pressures of ethylene gas, a N₂ carrier gas and a He carrier gas were each set at 33 kPa, and the contact time was set at 8.0 (g-catalyst·h/mol), and the reaction temperature was set at 350 to 500° C. FIG. 5 shows the ethylene conversion rate with time-dependent changes, and FIG. 6 shows the propylene selectivity with time-dependent changes.

As shown in FIG. 5, the ethylene conversion rate is high when the reaction temperature is in the rage of 400° C. to 500° C., and is particularly high at 430° C. However, at all reaction temperatures, the conversion rate declined over time.

As shown in FIG. 6, the propylene selectivity remained substantially the same in the range of reaction temperatures of 400° C. to 430° C. However, at a reaction temperature of 500° C. or higher, the propylene selectivity declined over time.

These results show that the reaction temperature of 400° C. to 500° C. achieves high values of the ethylene conversion rate and the propylene selectivity .

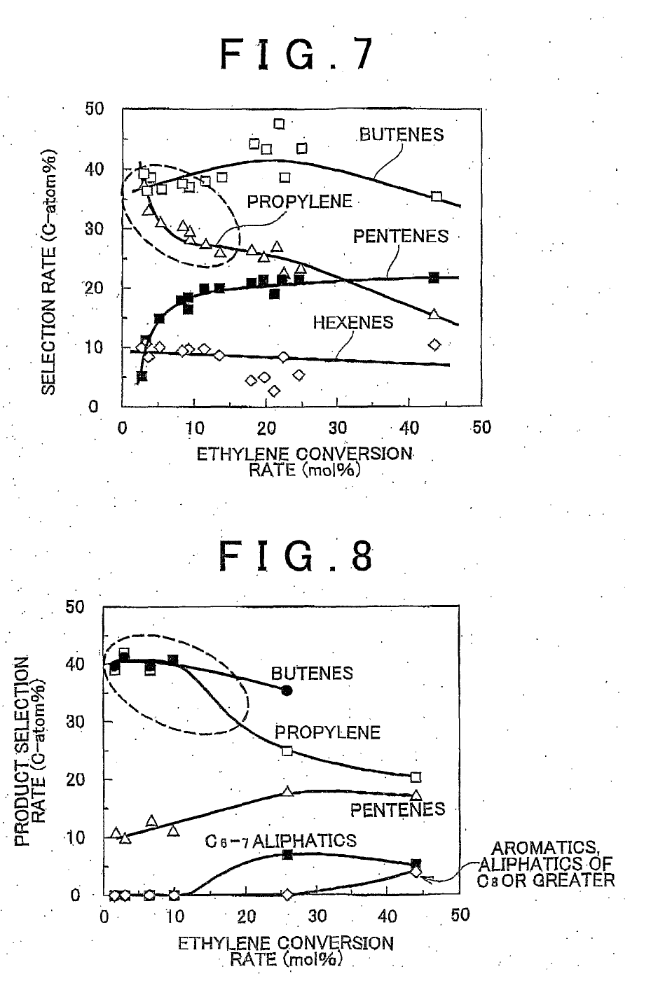

Hereinafter, the production test of propylene in a reaction system where water was removed by the ethanol dehydration reaction will be described. In this production test, H-ZSM-5 catalyst of SiO₂/Al₂O₃=23.8 was used. Ethanol and ethylene were used as starting raw materials. Test results are shown in FIGS. 7 and 8.

The reaction conditions are as follows. The pressures of ethanol and the carrier gas (He) were each set at 50 kPa, and the total amount of flow was set at 20 cm³/min, and the reaction temperature was set at 320° C. The reaction conditions after the dehydration reaction of ethanol are as follows, the pressures of ethylene gas, the N₂ carrier gas and the He carrier gas were each set at 33 kPa, and the total amount of flow was set at 17 cm³/min, and the reaction temperature was set at 320° C.

At 320° C., all of the ethanol underwent the dehydration reaction; thus all of the ethanol was consumed. Specifically, ethanol was converted into hydrocarbons. In FIG. 7, the horizontal axis represents the ethylene conversion rate. In the region indicated by a broken line in FIG. 7, the propylene selectivity sharply decreases in and around a range of ethylene conversion rate of 0 to 10 mol %. On the other hand, it was confirmed that the exposure of ethanol to the H-ZSM-5 catalyst (reaction temperature: 250° C.) resulted in the formation of ethylene with a yield of about 100%. The ethylene is formed through the dehydration of ethanol. FIG. 8 shows results obtained by conducting the aforementioned test in the reaction system where the eliminated water was removed. Results of the test show that the removal of water after the conversion from ethanol to ethylene inhibited the decrease in the propylene selectivity when the H-ZSM-5 catalyst was used, as shown in a region of a broken line in FIG. 8.

Hereinafter, the test of the regeneration of the catalyst will be described. As shown in the production test of propylene in the case where the reaction temperature was changed, the catalytic activity declined over time. Analysis of the catalyst after the reaction revealed that coke or a coke precursor was deposited in the catalyst. In order to restore the catalyst by removing the coke or the coke precursor, the heating treatment in the atmospheric air (an oxygen atmosphere) was conducted.

The condition of the regeneration was as follows. As for the catalyst, an SiAlPO-34 catalyst that had been used in the production test at the reaction temperature of 400° C. was used. The reaction atmosphere was of the atmospheric air (in an oxygen atmosphere), and the heating time was 3 hours. Two heating temperatures of 430° C. and 630° C. were set.

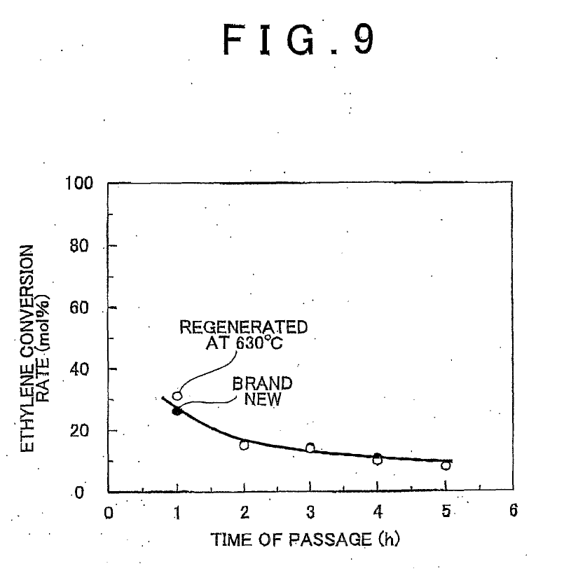

After the regeneration treatment, the propylene production test was conducted again. It was confirmed that in the catalyst subjected to the regeneration treatment at 430° C., the catalytic activity remained at the reduced level whereas in the catalyst subjected to the regeneration treatment at 630° C., the catalytic activity returned to the original level. Then, a test substantially the same as the aforementioned test was conducted, and changes in the ethylene conversion rate over time were observed. The reaction conditions are as follows. The pressures of the ethylene gas, the N₂ carrier gas and the He carrier gas were each set at 33 kPa, and the contact time was set at 8.0 (g-catalyst·h/mol), and the reaction temperature was set at 400° C. Test results are shown in FIG. 9.

As shown in FIG. 9, by subjecting the catalyst to the regeneration treatment at 630° C. significant catalytic activity was recovered such that the recovered catalyst had activity that was substantially equal to that of an unused catalyst (brand new). The decline of the catalytic activity is mainly caused by deposition of coke or a coke precursor. Therefore, by performing the catalyst regeneration treatment under the described conditions, the coke or the coke precursor is removed. Thus, the catalytic activity is easily recovered.

While the invention has been described with reference to example embodiments thereof, it should be understood that the invention is not limited to the described embodiments or constructions. To the contrary, the invention is intended to cover various modifications and equivalent arrangements. In addition, while the various elements of the described embodiments are shown in various combinations and configurations, other combinations and configurations, including more, less or only a single element, are also within the spirit and scope of the invention.

1. A production method for propylene, wherein:
ethanol is converted into propylene by continuously reacting ethanol on a catalyst;
the catalyst is a solid acid catalyst; and
an aperture diameter of pores formed in the solid acid catalyst is 0.3 to 0.5 nm.

2. The production method according to claim 1, wherein the ethanol is converted into the propylene by:
forming ethylene through a dehydration reaction between the catalyst and the ethanol;
forming a butyl cation through a dimerization reaction between the ethylene formed and an ethyl cation obtained from the catalyst;
forming a butene from the butyl cation;
forming a carbocation through a trimerization reaction between the butene and the ethyl cation; and
forming propylene through a cleavage reaction of the carbocation.

3. (canceled)

4. The production method for propylene according to claim 1, wherein a kinetic constant k of a butane cracking reaction of the solid acid catalyst is 0.1 to 30 (cm³/min·g).

5. The production method for propylene according to claim 4, wherein the solid acid catalyst is a phosphate-based zeolite.

6. The production method for propylene according to claim 5, wherein the phosphate-based zeolite is used and a reaction temperature is 300 to 500° C.

7. The production method for propylene according to claim 1, wherein the solid acid catalyst is doped with at least one species of metal ion selected from the group consisting of alkali metals, alkaline-earth metals, Group 5 metals, Group 8 metals, Group 10 metals, Group 11 metals and Group 12 metals.

8. The production method for propylene according to claim 1, wherein the solid acid catalyst is doped with lead ions.

9. The production method for propylene according to claim 1, wherein the solid acid catalyst is doped with calcium ions.

10. The production method for propylene according to claim 1, wherein the solid acid catalyst is doped with zinc ions.

11. The production method for propylene according to claim 1, wherein the catalyst into which the metal ion has been introduced is used as the solid acid catalyst, and a reaction temperature is 300 to 500° C.

12. (canceled)

13. The production method for propylene according to claim 1, wherein, when the ethanol is continuously converted into the propylene, a solid catalyst bed charged with at least one species of the solid acid catalyst is disposed at a side opposite from a side where the ethanol is supplied, and a dehydration catalyst bed charged with a dehydration catalyst is disposed at an upstream side of the solid acid catalyst bed.

14. The production method for propylene according to claim 13, wherein the moisture removal bed is disposed between the dehydration catalyst bed and the solid acid catalyst bed.

15. A regeneration method for a catalyst, characterized in that a heating treatment in an oxygen atmosphere is perform on a catalyst that has been used to produce propylene in the production method for propylene according to claim 1.

16. A solid acid catalyst characterized in that a kinetic constant k of a butane cracking reaction on the solid acid catalyst at 500° C. is 0.1 to 30 (cm³/min·g), and the solid acid catalyst is used in the production method for propylene according to claim 1.

17. A solid acid catalyst characterized in that an aperture diameter of pores of the solid acid catalyst is 0.3 to 0.5 nm, and the solid acid catalyst is used in the production method for propylene according to claim 1.
